(19) United States
(12) Patent Application Publication
(10) Pub. No.: US 2018/0116543 A1
(43) Pub. Date: May 3, 2018

(54) SYSTEM AND METHOD OF BRAIN EVENT CAPTURE MONITORING AND EVALUATING WITH RESPECT TO ACCUMULATED HISTORIES

(71) Applicants: LANDON C.G. MILLER, Tuscaloosa, AL (US); SCOTT BEHRENS, Noblesville, IN (US); KEVIN BUTTERFIELD, Noblesville, IN (US)

(72) Inventors: LANDON C.G. MILLER, Tuscaloosa, AL (US); SCOTT BEHRENS, Noblesville, IN (US); KEVIN BUTTERFIELD, Noblesville, IN (US)

(21) Appl. No.: 15/348,114

(22) Filed: Nov. 10, 2016

Related U.S. Application Data
(60) Provisional application No. 62/417,025, filed on Nov. 3, 2016.

(51) Int. Cl. A61B 5/0484; A61B 5/04; A61B 5/00

(52) U.S. Cl. CPC A61B 5/0484; A61B 5/04012; A61B 5/002; A61B 2562/0261; A61B 5/4064; A61B 2562/0219; A61B 5/0022

(57) ABSTRACT

A method for diagnosing and rapidly introducing treatment to a subject suffering visible or non-visible traumatic brain injury is provided. A wearable array of electroencephalography (EEG) sensors in electrical communication with a communications capability to one or more subjects is present. EEG readings from the one or more subjects in a database are accumulated. The occurrence of an accident or other stress forces experienced by a wearer that mimics a concussion or other trauma to the wearer's brain is detected. Based on a comparative analysis of the stored EEG readings in the database a determination is made whether a traumatic brain injury has occurred. A system for diagnosing and the rapid treatment of a wearer suffering traumatic brain injuries according to the inventive method is also provided.

### RELATED APPLICATIONS

This application is a non-provisional application that claims priority benefit to U.S. Provisional Application Ser. No. 62/417,025 filed 3 Nov. 2016; the contents of which are hereby incorporated by reference.

### FIELD OF THE INVENTION

The present invention in general relates to the field of medicine and in particular to a system for implementing real time brain event capture and evaluation for a course of treatment based on accumulated historical electroencephalography readings.

### BACKGROUND OF THE INVENTION

Traumatic brain injury (TBI), also known as intracranial injury, occurs when an external force traumatically injures the brain. TBI is a major cause of death and disability worldwide, especially in children and young adults. Causes of TBI include falls, vehicle accidents, sports injuries, and violence. Prevention measures include the use of technology to protect those suffering from automobile accidents, such as seat belts, caps, and helmets for sporting participants or motorcycle users.

Brain trauma may occur as a consequence of a focal impact upon the head, by a sudden acceleration/deceleration within the cranium or by a complex combination of both movement and sudden impact. In addition to the damage caused at the moment of injury, brain trauma causes secondary injury, a variety of events that take place in the minutes and days following the injury. These processes, which include alterations in cerebral blood flow and the pressure within the skull, contribute substantially to the damage from the initial injury. Thus, the speed at which treatment is begun can have a substantial and positive impact for mitigating the long-term effects of TBI. FIG. 1 shows a general timeline in the form of brain trauma windows of amelioration opportunities for treatment of TBI.

The recognition of traumatic brain injuries especially in cap or helmet wearing sports such as American football and motor racing, and in military, e.g. explosions, and many civilian activities in which shock and strikes to the head occur, has increasingly been recognized in recent years, as well as the need to speedily treat these injuries. As further examples, the detection of these events in such head involved sports such as soccer and Rugby, and sports where a head could be struck by a flying object such as ice and/or field hockey and baseball also has been increasing. Construction, workplace and other areas of potential head injury occurrence are also circumstances in which such early detection can have a salutary effect. Essentially, there exist a need for improved devices and methods for diagnosing and rapidly introducing treatment to patients suffering traumatic brain injuries either obvious or non-obvious from many different sources of head strikes and abrupt changes in the head's physical attitude.

### SUMMARY OF THE INVENTION

A method for diagnosing and rapidly introducing treatment to a subject suffering visible or non-visible traumatic brain injury is provided. A wearable array of electroencephalography (EEG) sensors in electrical communication with a communications capability to one or more subjects is present. EEG readings from the one or more subjects in a database are accumulated. The occurrence of an accident or other stress forces experienced by a wearer that mimics a concussion or other trauma to the wearer's brain is detected. Based on a comparative analysis of the stored EEG readings in the database a determination is made whether a traumatic brain injury has occurred.

A system for diagnosing and the rapid treatment of a wearer suffering traumatic brain injuries is also provided that includes a wearable array of electroencephalography (EEG) sensors in electrical communication with a processor and a communications capability to the head of wearer or a plurality of subjects. A database that accumulates and stores EEG readings from the wearer or a plurality of subjects with previously collected, comparative or normative sensor readings, or key elements of these readings, being down-loaded to the wearable processor system for subsequent real-time comparison.

### BRIEF DESCRIPTION OF THE DRAWINGS

The present invention is further detailed with respect to the following drawings. These figures are not intended to limit the scope of the present invention but rather illustrate certain attributes thereof.

FIG. 1 is a block diagram showing the treatment stages for a traumatic brain injury;

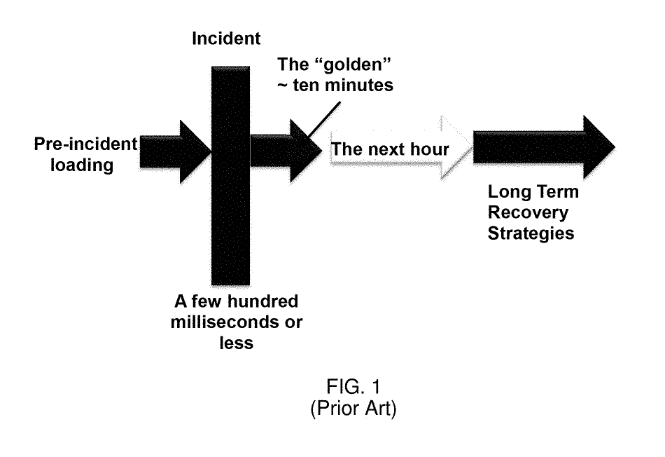

FIG. 2A is a placement map of electroencephalography (EEG)/quantitative electroencephalography (QEEG) sensor system overlaid on the regions of the human brain;

FIG. 2B is a color map of brainwave activity under concussion and normal brain state;

FIGS. 3A and 3B are photographs of an EEG/QEEG sensor system built into hood materials for implementing embodiments of the invention;

FIG. 4 is a partial perspective of a helmet with EEG/QEEG sensor system and a real-time process controlled cooling system with communication capabilities for introducing treatments to a wearer suffering a brain trauma injury according to an embodiment of the invention; and

FIG. 5 is a schematic diagram illustrating an overall view of communication devices, computing devices, and mediums for implementing embodiments of the invention.

### DESCRIPTION OF THE INVENTION

The present invention has utility as a method and system for diagnosing and rapidly introducing treatment to patients suffering visible and non-visible traumatic brain injuries based on real time brain event capture and evaluation for a course of treatment based on accumulated historical electroencephalography readings. Embodiments of the invention provide an integrated system of real time telemetry and active at-time-of-incident amelioration of potential and real forms of brain trauma. Embodiments of the invention are configured with a wearable map of the brain electroencephalography (EEG) points that may be worn in a headscarf, helmet, cap, or other headgear for providing real-time information on a wearer's brain condition. EEG is the recording of electrical activity along the scalp. EEG measures voltage fluctuations resulting from ionic current flows within the neurons of the brain. In an example specific embodiment, the EEG sensors are wired to a racing car vehicle communication system that transmits the brain wave data in real time of the driver. Other example embodiments include helmets, baseball type caps, and skull caps, each with EEG monitoring sensors and resultant data collection, analysis, and transmission components for either real-time or delayed transmission a part of an integrated, overall system. In embodiments of the invention, the overall system includes an application (app) or program software that may run on a tablet, portable communication device, or computing device on board the wearer portion of the system that processes the data from the EEG sensors and interprets the data in real time based on an accumulated history of EEG readings representing profiles, or patterns either in general, and/or for that specific wearer. In one specific embodiment, if there is an accident or other stress forces experienced by the wearer that mimics a concussion, the system initiates the real-time ejection of a cooling agent within the headgear to provide a net three to four-degree reduction in the victim's brain temperature with the helmet cooling system, and alerts a monitoring team of the situation. Embodiments of the invention may also include a drug intervention system kit and the injury detection app for expedited treatment of a potential brain injury during the critical early phases of the injury.

Embodiments of the brain injury tracking system collect event readings from the wearable map of the brain electroencephalography (EEG) points that may be worn in a headscarf, helmet, or other headgear, and captures key changes or a range of change in brain activity. The brain activity measurement may employ EEG sensors that are coupled to various processors and accelerometers with adequate memory and digital processing for analog to digital (A/D) conversion for radio frequency (RF) transmission via a dedicated device or a handheld device illustratively including a cell phone, smartphone, and tablet. In a specific embodiment, a communication plug-in device may be used with the cell phone, smartphone, or tablet to receive the brain wave information from the brain wave measuring headgear. Wireless communication protocols, illustratively including Bluetooth, nearfield communication, and WiFi, may be used to transfer brain wave information from the brain wave measuring headgear to a transmission device. In a specific embodiment, the brain wave measuring headgear may also be equipped with an RF transmitter to directly communicate brain wave data to the brain injury tracking system.

In inventive embodiments, the captured changes in brain activity may be transmitted to a remote database for storage and further analysis. In a specific embodiment, the database may be based in the cloud. Embodiments of the database record and store previous individual scans to form a baseline of a subject's brain activity, as well as storing event recordings related to occurrences of potential brain trauma. A collection of brain scans collected from a population of monitored subjects may be used to form normative population scan models. The normative models may be used to perform various analysis on received brain scan activity from individual subjects. One type of analysis that may be performed is causative correlation analysis. Causative correlations illustratively include analysis against historically accumulated normative normal brain activity from a population of subjects, as well as concussive events, and other event patterns from the population of subjects. The causative correlation may also be conducted based on individual patterns from a single subject.

Models of brain wave activity in embodiments of the database may illustratively include the following: standard EEG models by person type, concussive and other event diagnostic models, individual participant baseline models, and after event reconciliation models for use with the individual participant baseline models. Machine learning and artificial intelligence may be used to identify brain wave patterns and make diagnoses based on previously stored diagnoses made by physicians. A confidence level of machine made diagnoses may be established with a human review and accuracy grading of the machine-made diagnoses.

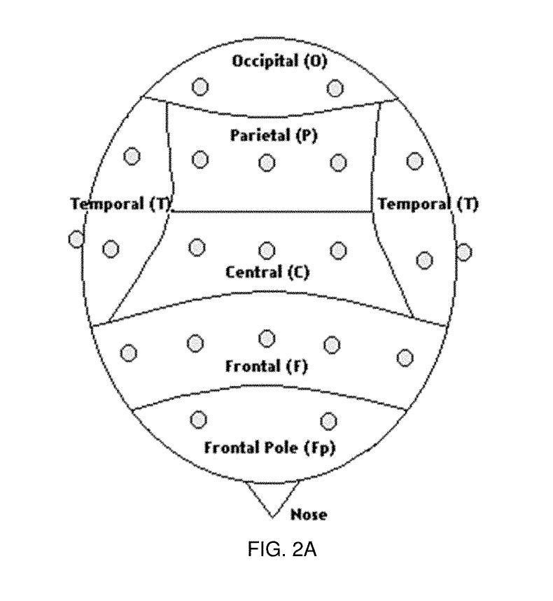

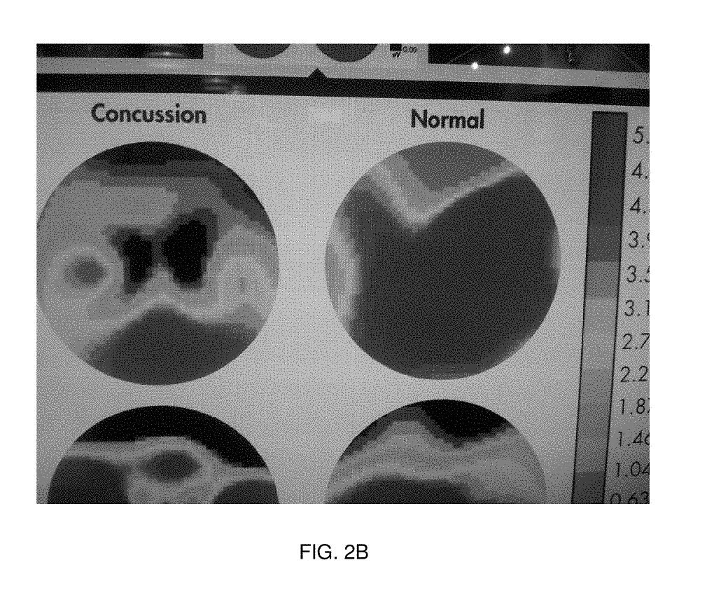

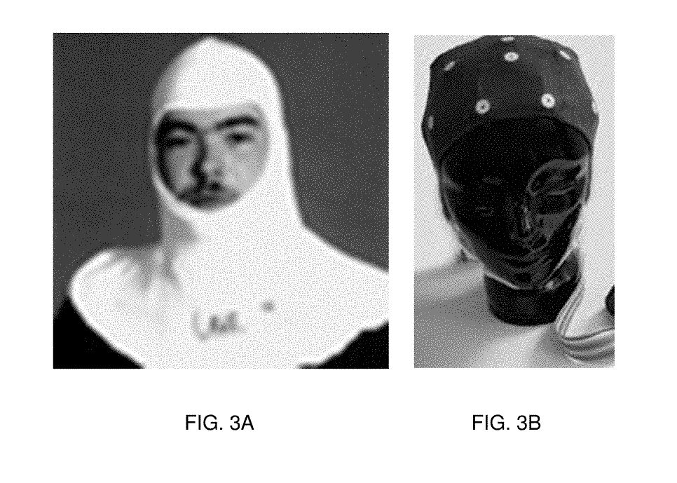

Referring now to the figures, FIG. 2A is a placement map of electroencephalography (EEG)/quantitative electroencephalography (QEEG) sensor array system overlaid on the regions of the human brain. FIG. 2B is a color map of brainwave activity under concussion and normal brain state as provided by the EEG sensor system. As shown in FIGS. 3A and 3B, the array of EEG sensors may be built into headwear, hoods, or headscarves that may be made of flame-resistant meta-aramid materials such as Nomex™ or other such materials. In addition, the EEG sensors may be built into helmet strapping, helmet cushions, or both, depending upon the application. Illustrative examples of helmets include football, baseball, skiing, bicycle, police, security forces, construction, and other application that require wearer head protection.

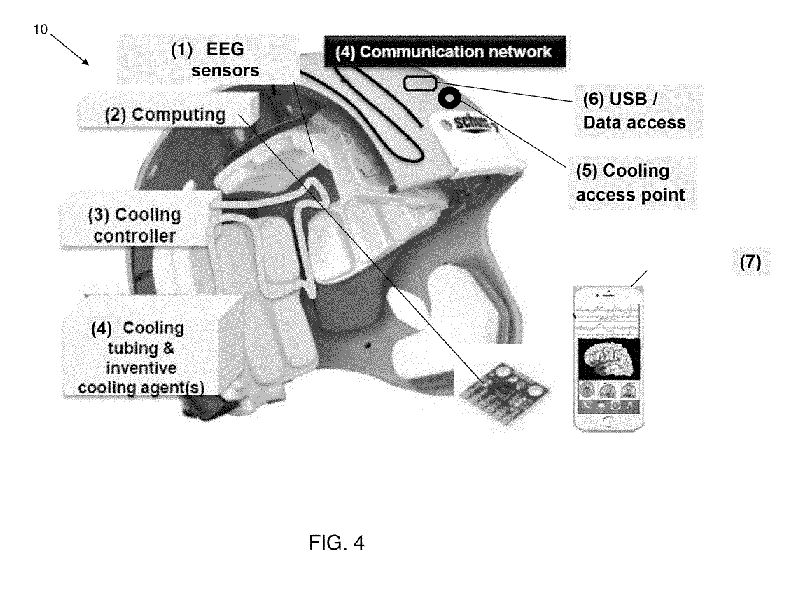

FIG. 4 is a partial perspective of a helmet 10 with EEG/QEEG sensor system (1) and a real-time process controlled cooling system (3, 4) with communication capabilities (4) for introducing treatments to a wearer suffering a brain trauma injury based on comparison of measured brain wave activity to stored models and brain wave histories. Embodiments of the helmet 10 have the EEG sensors (1) built into the helmet strapping, helmet cushions, or both, depending upon the application, with a computing processor (2) and associated electronics including a set of, depending upon the application, accelerometers, strain gauges, gyroscopes, force sensors, and other such sensors to determine, in real time, the actions that are occurring as an incident to a wearer occurs. It is appreciated that a conventional smart phone contains the computation and display functions to perform the attributes of the present invention when coupled with a helmet 10. The computing processor controls a real-time process control cooling system that includes tubing holding unmixed or mixed coolant(s) (e.g., inventive entropic lipids, etc.) such that upon release of the coolants into the cooling tubing, the temperature of the center of the brain may have its temperature lowered from 2 to 4 degrees Fahrenheit. The temperature of the brain may be lowered in a time frame that ranges from some number of seconds to a couple of minutes, depending upon severity of the impact to the helmet, and other factors, with the lowered temperature maintained for at least an hour. The cooling system formed with the cooling tubing may be integrated into the padding or mesh in the headwear or helmet. In a specific embodiment, an external supply of coolant may be attached to the headwear via a cooling access point (5). Embodiments of the helmet may also have a built-in communication system for two-way data transmission of sufficient speed and bandwidth for data collection, analysis, and directive actions to take place in real-time. Communication may take place utilizing a wired universal serial bus (USB) or other wired connection port (6), or via wireless data transfer via Wi-Fi/Bluetooth/nearfield/cellular/digital radio, etc. Computing systems (7) and devices illustratively including a smart phone, tablet, desktop computer, laptop, or portable computing device, may inter-connect to the cooling and the EEG sensor systems.

In embodiments, the EEG data collected may be analyzed and observations and recommendations may be made in real time including: type of injury predicted based on impact force; hookup of peripheral damage detection system; patient movement precautions; and interventional neuro-protective drug strategies to be introduced based on the EEG readings and locations of the brain injury. A graphical user interface (GUI) may be used to present the observations and recommendations.

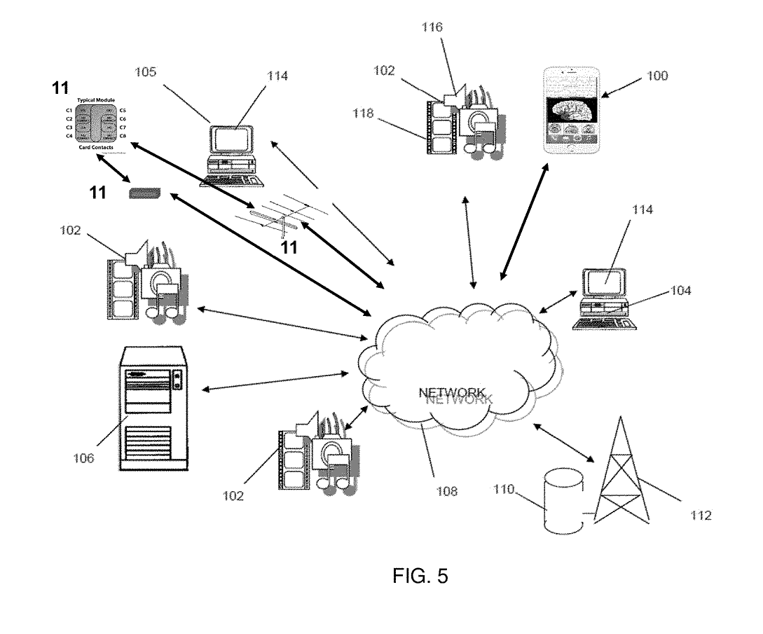

FIG. 5 is a schematic diagram illustrating an overall view of communication devices, computing devices, and mediums for implementing a system and method for diagnosing and rapidly introducing treatment to patients suffering traumatic brain injuries based on real time brain event capture and evaluation for a course of treatment based on accumulated historical electroencephalography readings.

The system 100, such as one loaded onto a smart phone, includes multimedia devices 102 and desktop computer devices 104 configured with display capabilities 114 and processors for executing instructions and commands. The multimedia devices 102 are optionally mobile communication and entertainment devices, such as cellular phones, tablets, and mobile computing devices that in certain embodiments are wirelessly connected to a network 108. The multimedia devices 102 typically have video displays 118 and audio outputs 116. It is appreciated that the information is readily communicated to a card connects module 115 of a contact and contactless memory smart card.

The multimedia devices 102 and desktop computer devices 104 are optionally configured with internal storage, software, and a graphical user interface (GUI) for carrying out elements of the brain wave tracking and diagnostic database platform according to embodiments of the invention. The network 108 is optionally any type of known network including a fixed wire line network, cable and fiber optics, over the air broadcasts, satellite, local area network (LAN), wide area network (WAN), global network (e.g., Internet), intranet, etc. with data/Internet capabilities as represented by server 106. Communication aspects of the network are represented by cellular base station 110 and antenna 112. In a preferred embodiment, the network 108 is a LAN and each remote device 102 and desktop device 104 executes a user interface application (e.g., Web browser) to contact the server system 106 through the network 108. Alternatively, the remote devices 102 and 104 may be implemented using a device programmed primarily for accessing network 108 such as a remote client. Brain wave monitors 122 may connect with the network 108 or directly to multimedia devices 102 and desktop computer devices 104 to provide EEG readings for storage in database 120.

The software for the brain wave tracking and diagnostic database platform, of embodiments of the invention, may be resident on tablets 102, desktop or laptop computers 104, or stored within the server 106 or cellular base station 110 for download to an end user. Server 106 may be implemented as a cloud-based service for implementing embodiments of the platform with the database 120 representing a multi-tenant database for storage of separate client data for each independent medical facility or office using the platform.

The foregoing description is illustrative of particular embodiments of the invention, but is not meant to be a limitation upon the practice thereof. The following claims, including all equivalents thereof, are intended to define the scope of the invention.

1. A method for diagnosing and rapidly introducing treatment to a wearer suffering visible or non-visible traumatic brain injury (TBI), said method comprising:
providing a wearable array of electroencephalography (EEG) sensors in electrical communication with a communications capability to one or more subjects;
accumulating and storing EEG readings from the one or more subjects in a database;
detecting whether an accident or other stress forces experienced by a wearer that mimics a concussion or other trauma to the wearer's brain based on sensors being worn by the wearer; and
determining whether a traumatic brain injury has occurred to the wearer based on a comparative analysis of the stored EEG readings in the database.

3. The method of claim 1 further comprising forming a baseline for the wearers individual brain activity the accumulated and stored EEG readings.

4. The method of claim 1 further comprising forming normative population scan models from the accumulated and stored EEG readings.

5. The method of claim 4 further comprising performing causative correlation analysis based on the normative population scan models.

6. The method of claim 4 wherein the normative population scan models further comprise at least one of: standard EEG models by person type, concussive and other event diagnostic models, individual subject baseline models, and after event reconciliation models for use with the individual subject baseline models.

7. The method of claim 1 further comprising using machine learning and artificial intelligence to identify brain wave patterns and make diagnoses based on a set of previously stored diagnoses made by physicians.

8. The method of claim 7 further comprising establishing a confidence level of machine made diagnoses with a human review and accuracy grading of the machine-made diagnoses.

9. The method of claim 1 wherein the wearable array of electroencephalography (EEG) sensors are in electrical communication with a processor that controls a cooling mechanism that cools the brain of the wearer from 2 to 4 degrees Fahrenheit in response to a detected accident or stress event.

10. The method of claim 9 wherein the temperature of the brain is lowered in a time frame that ranges in some seconds to a couple of minutes, depending upon severity of the accident or other stress forces experienced by the wearer.

11. The method of claim 1 further comprising detecting the stress forces in real time with at least one of accelerometers, strain gauges, gyroscopes, or force sensors, as an incident to the wearer occurs.

12. The method of claim 1 wherein said communications capability provides two-way data transmission utilizing at least one of Wi-fi, Bluetooth, cellular, digital radio, or near field communications technologies.

13. The method of claim 1 further comprising analyzing a set of data collected from said wearable array of electroencephalography (EEG) sensors and making observations and recommendations including: type of injury predicted based on stress force, hookup of a peripheral damage detection system, patient movement precautions, and interventional neuro-protective drug strategies to be introduced based on the EEG readings and locations of a brain injury.

14. A system for diagnosing and the rapid treatment of a wearer suffering traumatic brain injuries, said system comprising:
a wearable array of electroencephalography (EEG) sensors in electrical communication with a processor and a communications capability on the head of the wearer or a plurality of subjects; and
a database that accumulates and stores EEG readings from the wearer or the plurality of subjects; and where previously collected, comparative or normative sensor readings, or key elements of these readings, are down-loaded to the wearable processor system for subsequent real-time comparison.

15. The system of claim 14 further comprising a cooling mechanism with a coolant in a series of tubes of the cooling mechanism to cool the wearer's brain in response to a detected accident or stress event wherein the wearer's brain is cooled from 2 to 4 degrees Fahrenheit.

16. The system of claim 14 wherein said wearable array is built into a helmet, headwear, hoods, or headscarves.

17. The system of claim 14 wherein a set of stress forces resulting from the detected accident or stress event are detected in real time with at least one of accelerometers, strain gauges, gyroscopes, or force sensors as an incident to the wearer occurs.

18. The system of claim 14 wherein said communications capability provides two-way data transmission utilizing at least one of Wi-fi, Bluetooth, cellular, or digital radio.

19. The system of claim 14 wherein the database is cloud based.
